(19) United States
(12) Patent Application Publication
(10) Pub. No.: US 2007/0191904 A1
(43) Pub. Date: Aug. 16, 2007

(54) EXPANDABLE STIMULATION ELECTRODE WITH INTEGRATED PRESSURE SENSOR AND METHODS RELATED THERETO

(76) Inventors: Imad Libbus, St. Paul, MN (US); Jeffrey E. Stahmann, Ramsey, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/276,107

(22) Filed: Feb. 14, 2006

(51) Int. Cl. A61N 1/00

(52) U.S. Cl. 607/44

(57) ABSTRACT

This patent document discusses, among other things, apparatuses and methods including an expandable stimulation electrode with an integrated pressure sensor. In various examples, the apparatus further comprises a pulse generator, a controller, a posture sensor, or a physiological parameter sensor. When expanded, the electrode is adapted to abut a wall of a pulmonary artery, thereby providing an arterial anchor for the integrated pressure sensor. In addition, the expandable electrode provides a means to deliver baroreflex stimulation signals, generated by the pulse generator, to one or more baroreceptors in the arterial wall. Based on pressure sensor-provided signals indicative of an arterial blood pressure, the controller provides stimulation instructions to the pulse generator. The posture sensor may be used to normalize the pressure data or limit such data collection to a single posture orientation. In one example, the physiological parameter sensor includes a temperature sensor.

### CROSS-REFERENCE TO RELATED APPLICATIONS

The following commonly assigned U.S. patent applications are related and are all herein incorporated by reference in their entirety: “BAROREFLEX STIMULATION SYSTEM TO REDUCE HYPERTENSION,” Ser. No. 10/746,134, (Attorney Docket No. 279.675U.S.1); and “LEAD FOR STIMULATING THE BARORECEPTORS IN THE PULMONARY ARTERY,” Ser. No. 10/746,861, (Attorney Docket No. 279.694U.S.1).

### TECHNICAL FIELD

This patent document pertains generally to medical devices. More particularly, but not by way of limitation, this patent document pertains to systems, apparatuses, and methods for reducing hypertension or other cardiovascular disorders using baroreceptor stimulation.

### BACKGROUND

Hypertension is a cause of heart disease and other related cardiac co-morbidities. Hypertension occurs when blood vessels constrict. As a result of the constricting, the heart must work harder to maintain flow at a higher blood pressure, which can contribute to heart failure (i.e., a clinical syndrome in which cardiac function causes a below normal cardiac output that can fall below a level adequate to meet the metabolic demand of peripheral tissues). A large segment of the general population, as well as a large segment of patients implanted with (for example) pacemaker or defibrillators, suffer from hypertension. The long-term mortality, as well as the quality of life, can be improved for this population if blood pressure and thus, hypertension, can be reduced. Many patients who suffer from hypertension do not respond to treatment, such as treatments related to lifestyle changes and hypertension drugs.

A pressoreceptive region or field is capable of sensing changes in pressure, such as changes in blood pressure. Pressoreceptor regions within a human are referred to as baroreceptors, and generally include any sensors of pressure changes. For example, baroreceptors include afferent nerves and further include sensory nerve endings that are sensitive to the stretching of an arterial or other vessel wall that results from increased blood pressure from within the corresponding vessel, and function as the receptor of a central reflex mechanism that tends to reduce the pressure.

Baroreflex functions as a negative feedback system, and relates to a reflex mechanism triggered by stimulation of one or more baroreceptors. Increased pressure stretches blood vessels, which in turn activates baroreceptors in the vessel walls. Activation of baroreceptors naturally occurs through internal (blood) pressure and corresponding stretching of the arterial or other vessel wall, causing baroreflex inhibition of sympathetic nerve activity (referred to as “SNA”) and a reduction in systemic arterial pressure. An increase in baroreceptor activity induces a reduction of SNA, which reduces blood pressure by decreasing peripheral vascular resistance.

### SUMMARY

An apparatus comprising an expandable stimulation electrode integrated with a pressure sensor. When expanded, the electrode is adapted to abut a wall of a pulmonary artery, thereby providing an arterial anchor for the integrated pressure sensor. In addition, the expandable electrode provides multiple contacts with the arterial wall to deliver baroreflex stimulation signals, generated by a pulse generator, to one or more baroreceptors located therein. Using signals indicative of an arterial blood pressure (provided, at least in part, by the pressure sensor), a controller provides one or more stimulation instructions to the pulse generator.

In various examples, the apparatus further comprises a posture sensor, a physiological parameter sensor, or a second electrode. The posture sensor may be used to normalize the (blood) pressure data or limit pressure data collection to a single posture orientation (e.g., recumbent). In one example, the physiological parameter sensor includes a temperature sensor providing pulmonary artery blood temperature information. In another example, the second electrode is positioned proximally from the expandable electrode to deliver a cardiac pacing signal also generated by the pulse generator.

A method comprising implanting an expandable electrode integrated with a pressure sensor within a pulmonary artery such that an outer surface of the electrode abuts a wall of the pulmonary artery, monitoring a signal indicative of a blood pressure in the pulmonary artery using the pressure sensor, and delivering a baroreflex stimulation signal to a baroreceptor in the pulmonary artery via the electrode is also discussed.

Various options for the method are possible. In one example, the method further comprises comparing the signal indicative of the pulmonary artery blood pressure with a predetermined pressure signal threshold. In another example, the method comprises modifying the baroreflex stimulation signal using the blood pressure indicative signal. In yet another example, the method comprises monitoring a signal indicative of a (subject's) then-current posture and normalizing the blood pressure indicative signal using the same.

These and other examples, aspects, advantages, and features of the apparatuses and methods described herein will be set forth, in part, in the Detailed Description that follows, and in part will become apparent to those skilled in the art by reference to the following description and drawings or by practice of the same.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various embodiments discussed in this patent document.

FIGS. 1A-1B illustrate neural mechanisms for peripheral vascular control.

FIGS. 2A-2C illustrate a heart or portions thereof.

FIG. 3 illustrates one or more baroreceptors and afferent nerves in the area of a carotid sinus and an aortic arch.

FIG. 4 illustrates one or more baroreceptors in or around a pulmonary artery.

FIG. 5 illustrates baroreceptor fields in an aortic arch, a ligamentum arteriosum, or a trunk of a pulmonary artery.

FIG. 6 illustrates a leadless apparatus comprising an expandable electrode integrated with a pressure sensor and a generalized environment in which the apparatus may be used, as constructed in accordance with at least one embodiment.

FIG. 7 illustrates a leaded apparatus comprising an expandable electrode integrated with a pressure sensor and a generalized environment in which the apparatus may be used, as constructed in accordance with at least one embodiment.

FIG. 8 illustrates an apparatus comprising an expandable electrode integrated with a pressure sensor coupled to an implantable medical device via a lead, as constructed in accordance with at least one embodiment.

FIG. 9 illustrates an implantable medical device, as constructed in accordance with at least one embodiment.

FIG. 10A illustrates a portion of a lead having an expandable electrode integrated with a pressure sensor coupled thereto, as constructed in accordance with at least one embodiment.

FIG. 10B illustrates the portion of the lead of FIG. 10A with the electrode integrated with a pressure sensor in an expanded configuration.

FIGS. 11A-11D illustrate an expandable electrode integrated with a pressure sensor, as constructed in accordance with various embodiments.

FIGS. 12A-12B illustrate a systematic overview of reducing hypertension using baroreceptor stimulation.

FIG. 13 illustrates a method of fabricating an apparatus comprising an expandable electrode integrated with a pressure sensor, as constructed in accordance with at least one embodiment.

FIG. 14 illustrates a method of using an apparatus comprising an expandable electrode integrated with a pressure sensor, as constructed in accordance with at least one embodiment.

### DETAILED DESCRIPTION

The following detailed description includes references to the accompanying drawings, which form a part of the detailed description. The drawings show, by way of illustration, specific embodiments in which the present apparatuses and methods may be practiced. These embodiments, which are also referred to herein as “examples,” are described in enough detail to enable those skilled in the art to practice the present apparatuses and methods. The embodiments may be combined or varied, other embodiments may be utilized or structural, logical, or electrical changes may be made without departing from the scope of the present apparatuses and methods. It is also to be understood that the various embodiments of the present apparatuses and methods, although different, are not necessarily mutually exclusive. For example, a particular feature, structure or characteristic described in one embodiment may be included within other embodiments. The following detailed description is, therefore, not to be taken in a limiting sense and the scope of the present apparatuses and methods are defined by the appended claims and their legal equivalents.

In this document the terms “a” or “an” are used to include one or more than one; the term “or” is used to refer to a nonexclusive or unless otherwise indicated; and the term “subject” is used to include the term “patient.” In addition, it is to be understood that the phraseology or terminology employed herein, and not otherwise defined, is for the purpose of description only and not of limitation. Further, by way of example, but not of limitation, the present apparatuses and methods are described for the most part with reference to a pulmonary artery location.

A brief discussion of hypertension and the physiology related to baroreceptors is provided below to assist the reader with understanding this patent document. This brief discussion introduces hypertension, the autonomic nervous system, and baroreflex.

Hypertension is a cause of heart disease and other related cardiac co-morbidities, and relates generally to high blood pressure, such as a transitory or sustained elevation of systemic arterial blood pressure at a level that is likely to induce cardiovascular damage or other adverse consequences. Hypertension has been arbitrarily defined as a systolic blood pressure above 140 mm Hg or a diastolic blood pressure above 90 mm Hg and occurs when blood vessels constrict. As a result of vessel constriction, a heart must work harder to maintain flow at a higher blood pressure. Consequences of uncontrolled hypertension include, but are not limited to, retinal vascular disease and stroke, left ventricular hypertrophy and failure, myocardial infarction, dissecting aneurysm, and renovascular disease.

The automatic nervous system (referred to as “ANS”) regulates “involuntary” organs, while the contraction of voluntary (skeletal) muscles is controlled by somatic motor nerves. Examples of involuntary organs include respiratory and digestive organs, and also include blood vessels and the heart. Often, the ANS functions in an involuntary, reflexive manner to regulate glands, to regulate muscles in the skin, eyes, stomach, intestines and bladder, and to regulate cardiac muscle and the muscle around blood vessels, for example.

The ANS includes, but is not limited to, the sympathetic nervous system and the parasympathetic nervous system. The sympathetic nervous system is affiliated with stress and the “fight or flight response” to emergencies. Among other effects, the “fight of flight response” increases blood pressure and heart rate to increase skeletal muscle blood flow, and decreases digestion to provide the energy for “fighting or fleeing.” The parasympathetic nervous system is affiliated with relaxation and the “rest and digest response” which, among other things, decreases blood pressure and heart rate, and increases digestion to conserve energy. The ANS maintains normal internal function and works with the somatic nervous system.

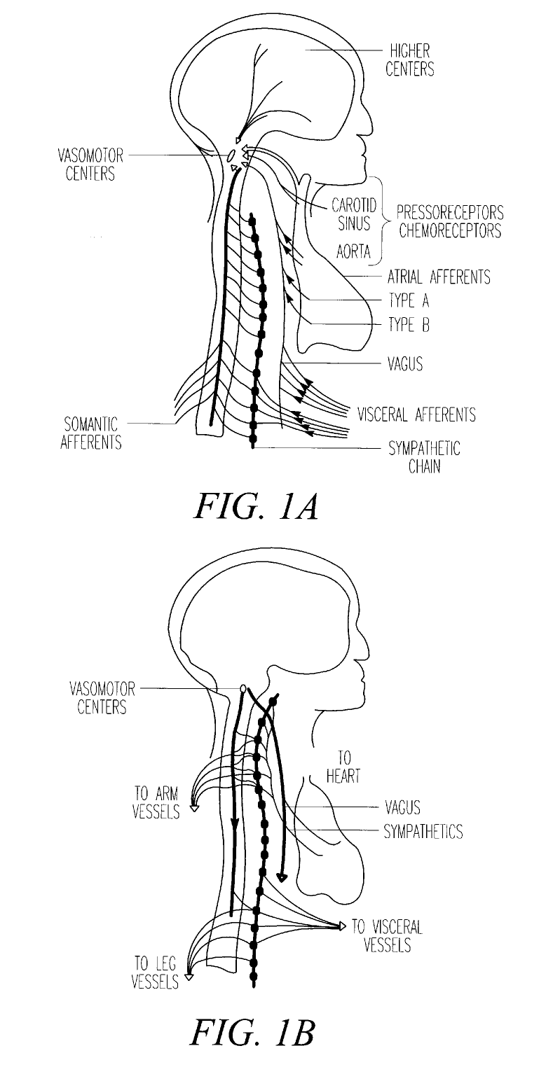

The subject matter of this patent document generally refers to the effects that the ANS has on the heart rate and blood pressure, including vasodilation and vasoconstriction. The heart rate and force is increased when the sympathetic nervous system is stimulated, and is decreased when the sympathetic nervous system is inhibited (e.g., when the parasympathetic nervous system is stimulated). FIGS. 1A-1B generally illustrate neural mechanisms for peripheral vascular control. Specifically, FIG. 1A illustrates the connection of afferent nerves to vasomotor centers. An afferent nerve conveys impulses toward a nerve center. A vasomotor center relates to nerves that dilate and constrict blood vessels to control the size of the blood vessels. FIG. 1B illustrates the connection of efferent nerves from vasomotor centers. An efferent nerve conveys impulses away from a nerve center.

Baroreflex is a reflex triggered by stimulation of one or more baroreceptors. A baroreceptor includes any sensor of pressure changes, such as sensory nerve endings in the wall(s) of the auricles of the heart, cardiac fat pads, vena cava, aortic arch or carotid sinus, that is sensitive to stretching of the wall resulting from increased pressure from within, and that functions as the receptor of the central reflex mechanism that tends to reduce that pressure. Additionally, a baroreceptor includes afferent nerve trunks, such as the vagus, aortic and carotid nerves, leading from the sensory nerve endings. Stimulating baroreceptors inhibits sympathetic nerve activity (stimulates the parasympathetic nervous system) and reduces systemic arterial pressure by decreasing peripheral vascular resistance. Baroreceptors are naturally stimulated by internal (blood) pressure and the stretching of one or more arterial walls.

Some aspects of the present apparatuses and methods locally and directly stimulate specific nerve endings in arterial walls rather than stimulate afferent nerve trunks in an effort to stimulate a desired response (e.g., reduced hypertension) while reducing the undesired effects of indiscriminate stimulation (e.g., pupil dilation or reduction of saliva and mucus production) of the nervous system. In one example, baroreceptor sites in the pulmonary artery are stimulated.

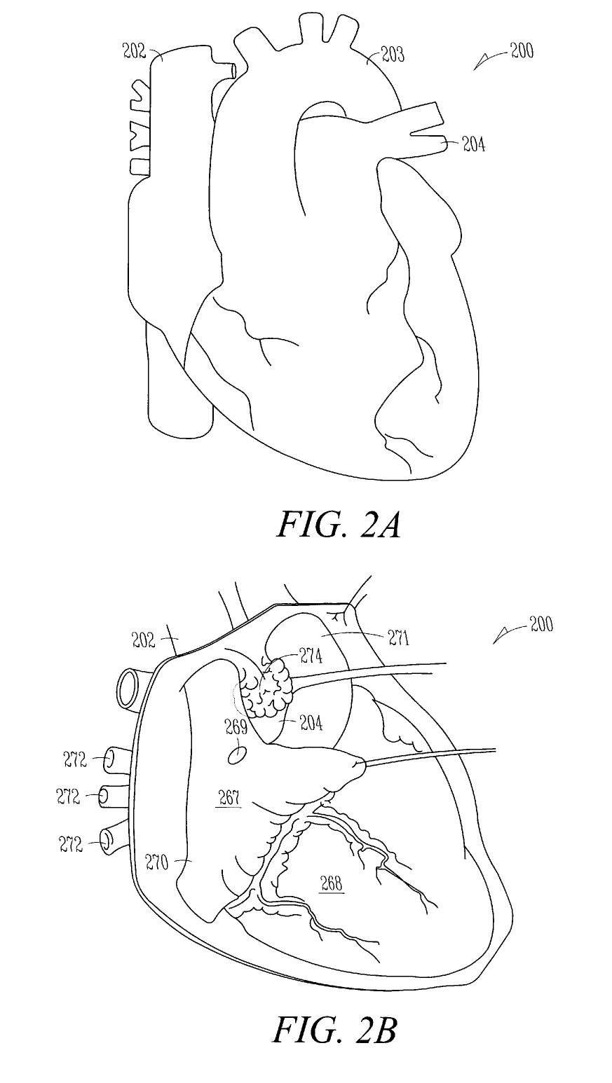

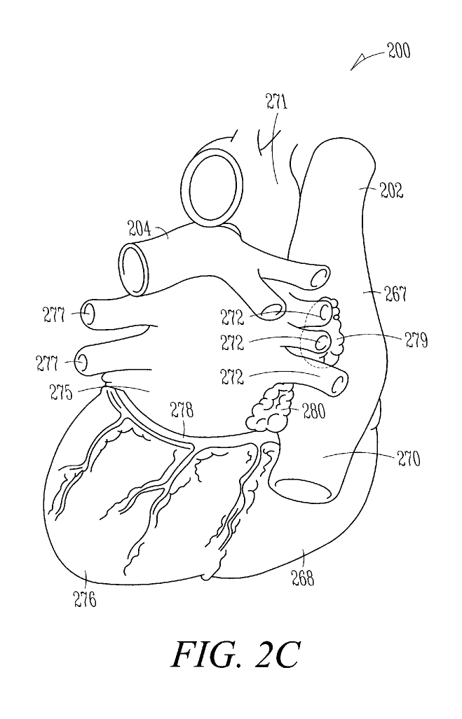

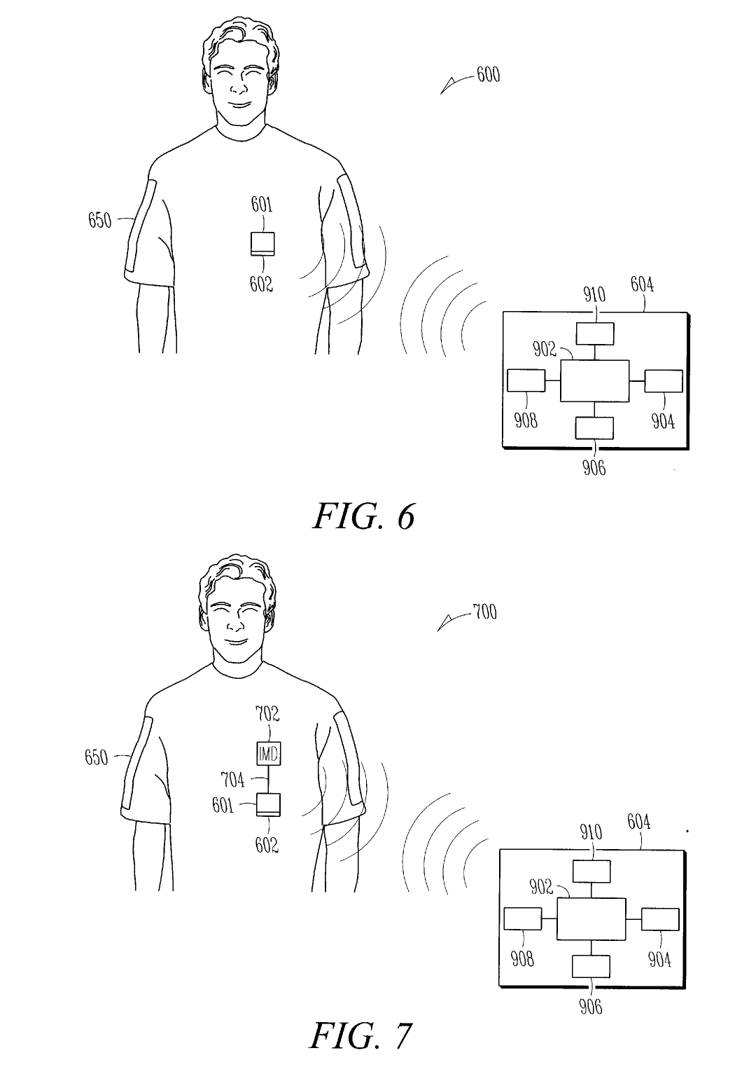

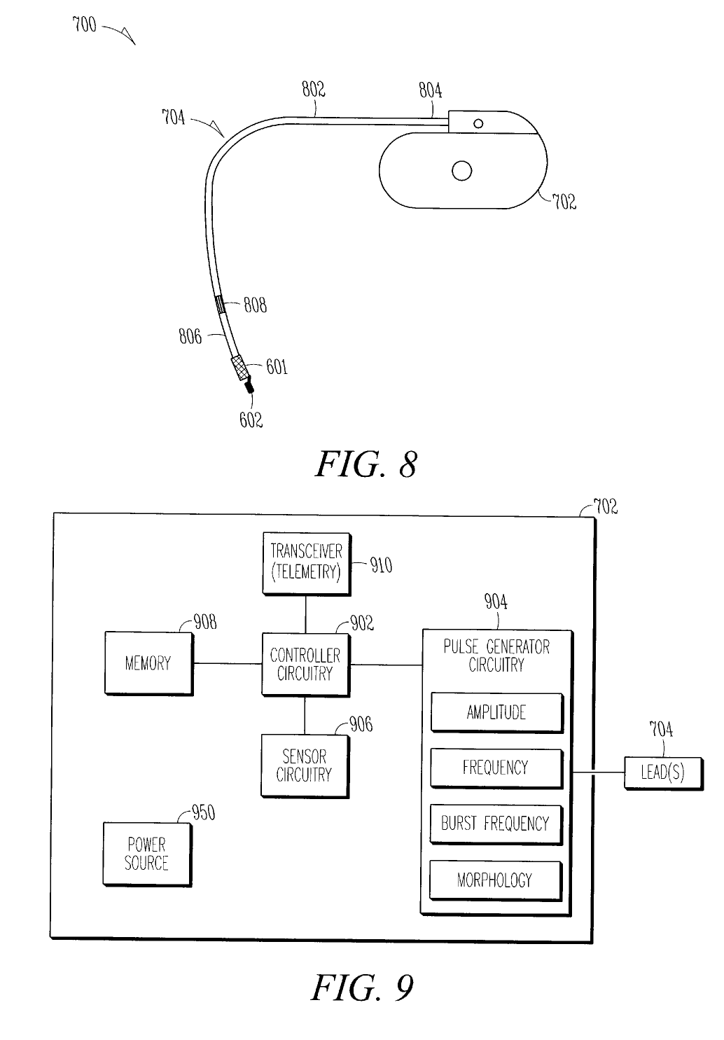

FIGS. 2A-2C illustrate a heart 200. As shown in FIG. 2A, heart 200 includes a superior vena cava 202, an aortic arch 203, and a pulmonary artery 204, all of which provide a useful contextual relationship for subsequent illustrations, such as FIGS. 3-5. As discussed below, pulmonary artery 204 includes one or more baroreceptors in a wall(s) thereof. Accordingly, a leaded (see, e.g., FIG. 7) or leadless (see, e.g., FIG. 6) apparatus comprising, among other things, an expandable electrode having an integrated pressure sensor may be disposed into a lumen of a pulmonary artery 204 for sensing and stimulation thereof. In one example, the leaded apparatus may be intravascularly inserted through a peripheral vein and a tricuspid valve into a right ventricle of heart 200 (not expressly shown in FIG. 2A), and continued from the right ventricle through the pulmonary valve into the lumen of pulmonary artery 204. In another example, the leadless apparatus may be positioned via a catheter into the lumen of pulmonary artery 204.

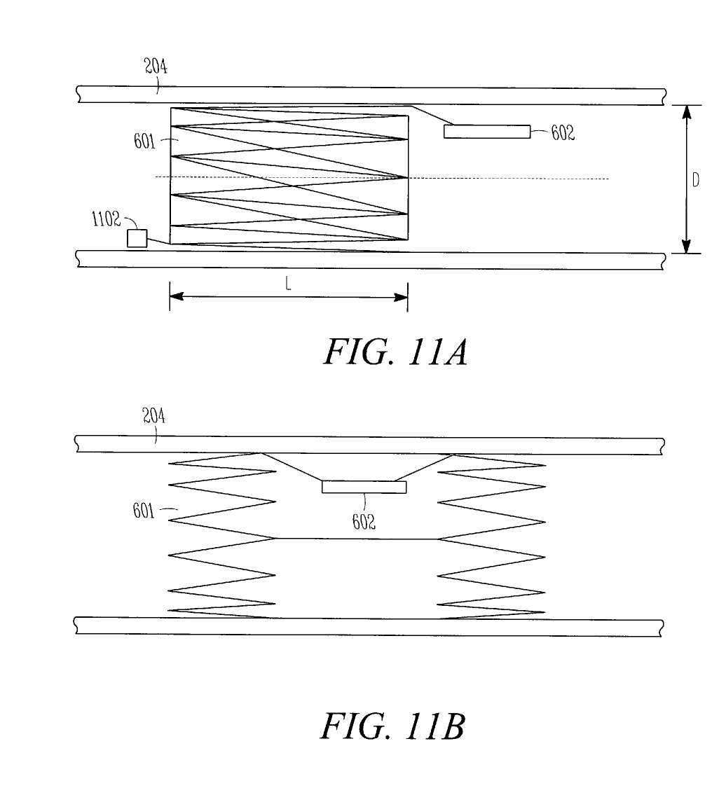

When positioned in pulmonary artery 204, the integrated pressure sensor may sense a signal indicative of an arterial (blood) pressure and communicate the same with a controller (see, e.g., FIG. 9). In one example, the controller compares the blood pressure indicative signal to a predetermined pressure threshold. If the blood pressure indicative signal is greater than the predetermined threshold, the leaded or leadless apparatus delivers one or more pulse generator-created stimulation pulses to baroreceptors located in a wall of pulmonary artery 204. In varying examples, control of the pulse generator (see, e.g., 9) is performed by the controller. In one such example, the controller is disposed in another implantable device, such as an implantable medical device (referred to as “IMD”) (see, e.g., FIG. 9). In another example, the controller is disposed in an external device (see, e.g., FIG. 6) and is adapted to communicate with the pulse generator via ultrasonic means, electromagnetic means, or a combination thereof.

FIGS. 2B-2C generally illustrate a right side and a left side of heart 200, respectively, and further illustrate one or more cardiac fat pads, which include nerve endings that (as discussed above) may function as baroreceptor sites. Specifically, FIG. 2B illustrates a right atrium 267, a right ventricle 268, a sinoatrial node 269, a super vena cava 202, an inferior vena cava 270, an aorta 271, one or more right pulmonary veins 272, and a pulmonary artery 204. In addition, FIG. 2B illustrates a cardiac fat pad 274 located between superior vena cava 202 and aorta 271. In one example, one or more baroreceptor nerve endings in cardiac fat pad 274 are stimulated using an electrode screwed into fat pad 274. In another example, the one or more baroreceptor nerve endings in cardiac fat pad 274 are stimulated using a leaded or leadless apparatus comprising an expandable electrode and integrated pressure sensor proximately positioned to fat pad 274 in a vessel, such as pulmonary artery 204 or superior vena cava 202.

FIG. 2C illustrates a left atrium 275, a left ventricle 276, a right atrium 267, right ventricle 268, superior vena cava 202, inferior vena cava 270, aorta 271, right pulmonary veins 272, a left pulmonary vein 277, pulmonary artery 204, and a coronary sinus 278. In addition, FIG. 2C illustrates a cardiac fat pad 279 located proximate to right cardiac veins 272 and a cardiac fat pad 280 located proximate to inferior vena cava 270 and left atrium 275. In one example, one or more baroreceptor nerve endings in fat pad 279 are stimulated using an electrode screwed therein. In another example, the one or more baroreceptor nerve endings in fat pad 279 are stimulated using a leaded or leadless apparatus comprising an expandable electrode and integrated pressure sensor proximately positioned to fat pad 279 in a vessel, such as pulmonary artery 204 or right pulmonary vein 272. One or more baroreceptors in cardiac fat pad 280 may be similarly stimulated using a leaded or leadless apparatus positioned in a vessel such as inferior vena cava 270, coronary sinus 278, or left atrium 275.

FIG. 3 illustrates a portion of a heart 200, including one more baroreceptors in the area of a carotid sinuses 305, an aortic arch 203 and a pulmonary artery 204. As shown, a vagus nerve 306 extends and provides sensory nerve endings 307 that function as baroreceptors in aortic arch 203, in carotid sinus 305, and in a common carotid artery 310. A glossopharyngeal nerve 308 provides nerve endings 309 that function as baroreceptors in carotid sinus 305. These nerve endings 307 and 309, for example, are sensitive to stretching of corresponding walls thereof resulting from increased pressure within. Activation of these nerve endings reduces pressure. Although not illustrated in FIG. 3, one or more fat pads, an atrial chamber, and a ventricular chamber of heart 200 also include baroreceptors.

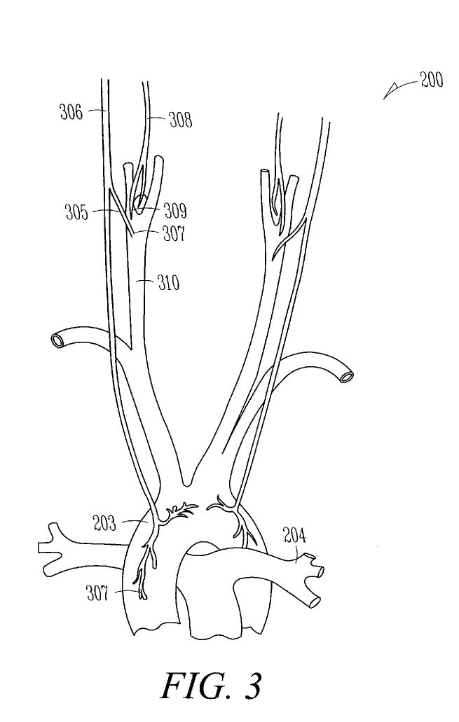

FIG. 4 illustrates baroreceptors in and around a pulmonary artery 204. As shown, pulmonary artery 204 includes one or more baroreceptors 411, as generally indicated by the circular-shaped regions. A cluster of closely spaced baroreceptors 411 is situated near the attachment of a ligamentum arteriosum 412. FIG. 4 also illustrates a superior vena cava 202, an aortic arch 203, a right ventricle 268 of heart 200 (FIG. 2A), and a pulmonary valve 414 separating right ventricle 268 from pulmonary artery 204. In one example, a leaded apparatus (including an expandable electrode integrated with a pressure sensor) is inserted through a peripheral vein and threaded through a tricuspid valve into right ventricle 268, and from right ventricle 268 through pulmonary valve 414 into pulmonary artery 202 to stimulate baroreceptors 411 in or around pulmonary artery 204. In one such example, the leaded apparatus is positioned to stimulate the cluster of baroreceptors near ligamentum arteriosum 412. In another example, a leadless apparatus (including an expandable electrode integrated with a pressure sensor) is positioned via a catheter into pulmonary artery 204.

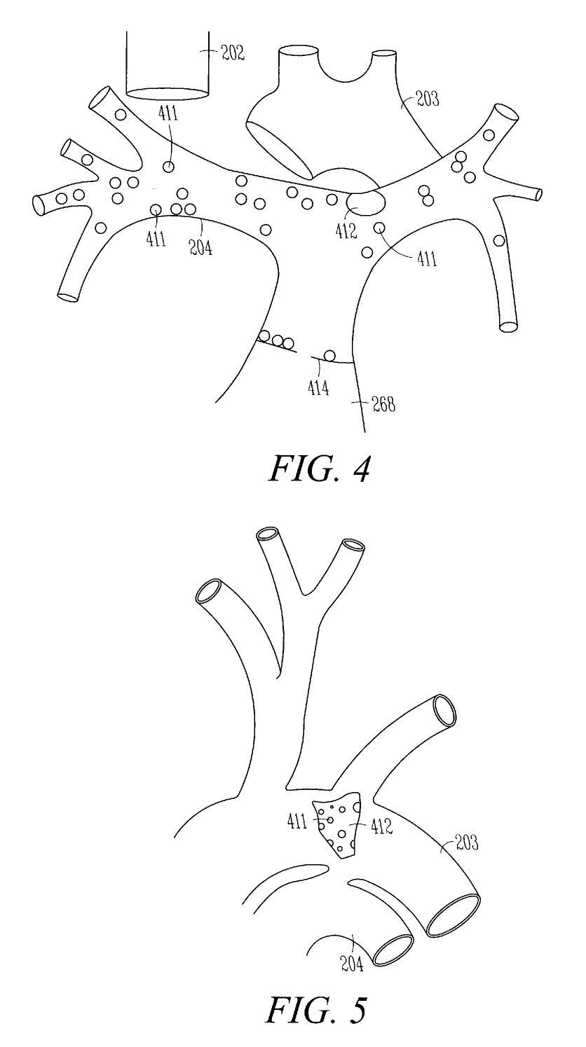

FIG. 5 illustrates one or more baroreceptors 411 in an aortic arch 203, near a ligamentum arteriosum 412 and a trunk of a pulmonary artery 204. In one example, a leaded or leadless apparatus (including an expandable electrode and an integrated pressure sensor) is positioned in pulmonary artery 204 to sense blood pressure therein and stimulate baroreceptors 411 in or around the arterial wall. In another example, the leaded or leadless apparatus is positioned in pulmonary artery 204 to stimulate baroreceptors 411 in aorta 271 or cardiac fat pads 274, such as are illustrated in FIG. 2B.

### EXAMPLES

The present apparatuses and methods relate to a chronically-implanted stimulation system specially designed to treat hypertension or other cardiovascular disorders (e.g., heart failure, coronary artery disease, etc.) by monitoring blood pressure and stimulating baroreceptors to activate the baroreceptor reflex and inhibit sympathetic discharge from the vasomotor center. In one example, the hypertension treatment is provided via a leaded apparatus including an expandable electrode and integrated pressure sensor coupled (via a lead) to another implantable device, such as an IMD (see, e.g., FIG. 7).

In one such example, the IMD includes both hypertension treatment elements (e.g., a high-frequency pulse generator, sensor circuitry to monitor posture or blood temperature, a controller, or a memory) and cardiac rhythm management (referred to as “CRM”) or advanced patient management (referred to as “APM”) components (e.g., components related to pacemakers, cardioverters/defibrillators, pacer/defibrillators, biventricular or other multi-site resynchronization or coordination devices, or drug delivery systems). Integrating hypertension treatment elements and CRM or APM components that are either performed in the same or separate devices improves aspects of the hypertension therapy (e.g., stimulation of one or more baroreceptors 411 (FIG. 4)) and cardiac therapy by allowing these therapies to work together intelligently, optionally in a closed-loop manner.

In another example, the hypertension therapy is provided via a stand-alone leadless apparatus including an expandable electrode and integrated pressure sensor (see, e.g., FIG. 6). In one such example, the leadless apparatus is capable of communicating with an external device wirelessly (e.g., ultrasonically or electromagnetically). The external device may include one or more hypertension treatment elements, CRM components, or APM components.

FIG. 6 illustrates a leadless apparatus 600 for treating, among other things, hypertension. In this example, apparatus 600 comprises an expandable electrode 601, a pressure sensor 602 integrated with electrode 601, and an external device 604. Expandable electrode 601 and integrated pressure sensor 602 are adapted to be implanted into a lumen of a pulmonary artery 204 (FIG. 4) and fixated to a wall thereof via the expandable nature of electrode 601.

After implantation, integrated pressure sensor 602 in association with sensor circuitry 906, measures a pulmonary artery (blood) pressure and provides a pressure indicative signal to a controller 902. Pressure sensor 602 and sensory circuitry 906 may be adapted to monitor pressure parameters such as mean arterial pressure, systolic pressure, diastolic pressure, or the like. In one example, as mean arterial pressure increases or remains above a predetermined target pressure (stored in, for example, memory 908), controller 902 directs a pulse generator 904 to deliver one or more stimulation pulses (e.g., about 5-10 seconds of stimulation each minute at a voltage of about 0.1-10 volts and a frequency between about 10-150 Hz) to baroreceptors located in a wall of pulmonary artery 204 thereby reducing blood pressure and controlling hypertension.

After baroreflex stimulation pulses have been applied, integrated pressure sensor 602 in association with sensor circuitry 906 may again generate a signal indicative of pulmonary artery (blood) pressure. Using the pressure indicative signal, controller 902 may modulate an amplitude, frequency, burst frequency, or morphology of the baroreflex stimulation pulses (see, e.g., FIG. 9). In one example, as the mean arterial pressure decreases toward the predetermined target pressure, controller 902 responses by instructing pulse generator 904 to deliver reduced baroreceptor stimulation or no stimulation at all.

In one example, one or more of controller 902, pulse generator 904, sensor circuitry 906, memory 908, and a transceiver 910 are included in external device 604 such as a Personal Digital Assistant (referred to as “PDA”) or personal laptop or desktop computer. In such an example, expandable electrode 601 and integrated pressure sensor 602 include a transceiver and associated circuitry for use to wirelessly communicate data and instructions with transceiver 910, and thus external device 604. Integrated pressure sensor 602 may thus, be programmed to deliver pulmonary artery (blood) pressure data to external device 604 at a fixed, predetermined time internal, or in response to a user-generated request thereby minimizing power consumption.

Leadless apparatus 600 may be powered in a variety of ways. In one example, apparatus 600 includes a capacitor (power source), which is ultrasonically or electromagnetically charged by an external unit, such as external device 604. In another example, integrated pressure sensor 602 includes a battery, which in one instance allows the sensor to transmit pressure data to external device 604 for 60 seconds per day for approximately 5 years.

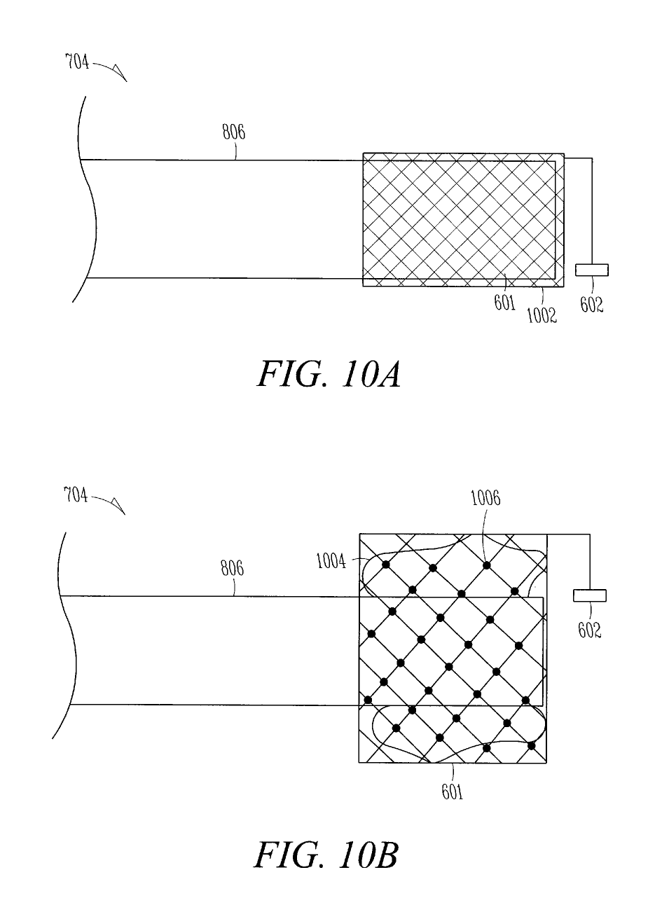

FIG. 7 illustrates a leaded apparatus 700 for treating, among other things, hypertension. In this example, apparatus 700 comprises an expandable electrode 601, a pressure sensor 602 integrated with electrode 601, an IMD 702, a lead 704, and an external device 604. Expandable electrode 601, integrated pressure sensor 602, IMD 702, and lead 704 are discussed in greater detail below in associated with FIGS. 8-9. External device 604, as discussed above in associated with FIG. 6, may include one or more of a memory 908, a transreceiver 910, a controller 902, sensor circuitry 906, or a pulse generator 904. In one example, external device 604 is an optional element as IMD 702 may contain all necessary hardware, circuitry, or software to perform the desired detection, processing, or therapy function(s). In another example, external device 604 alone or in combination with IMD 702 (via wireless communication) performs the desired detection, processing, or therapy function(s).

In both leadless 600 and leaded 700 apparatuses, a subject 650 may be provided with an external pressure reference (referred to as “EPR”) that he/she keeps with them (similar to how a subject typically keeps a cellular telephone or pager with him/her). The EPR functions as a trending barometer and makes barometric pressure measurements at predetermined times (e.g., once per minute). Data monitored by the EPR may be processed along with data from integrated pressure sensor 602 and sensor circuit 906 through the use of controller 902, for example. In this way, pulmonary artery (blood) pressure data is corrected for changes in barometric pressure. In one example, the EPR is included in a subject wearable device.

Further, as discussed above, both leadless 600 and leaded 700 apparatus may provide a combination of hypertension therapy and CRM or APM functions, which may optionally operate in a close-loop feedback manner. In one example, the hypertension treatment, CRM functions, or APM functions are capable of wirelessly communicating with each other (via programming in controller 902 or through the use of transreceiver 910). In one such example, an APM system includes an external blood pressure monitor, which is used for periodic calibration of integrated pressure sensor 602. In another such example, hypertension therapy (i.e., baroreceptor 411 (FIG. 4) stimulation) is modified using, among other things, one or more of electrophysiological parameters such as heart rate, minute ventilation, atrial activation, ventricular activation, or cardiac events collected by CRM or APM components. In addition, CRM components may modify therapy applied to (or about) a heart 200 (FIG. 2) based on data received from electrode 601 or integrated pressure sensor 602, such as mean arterial pressure, systolic and diastolic pressure, or baroreceptor stimulation rate.

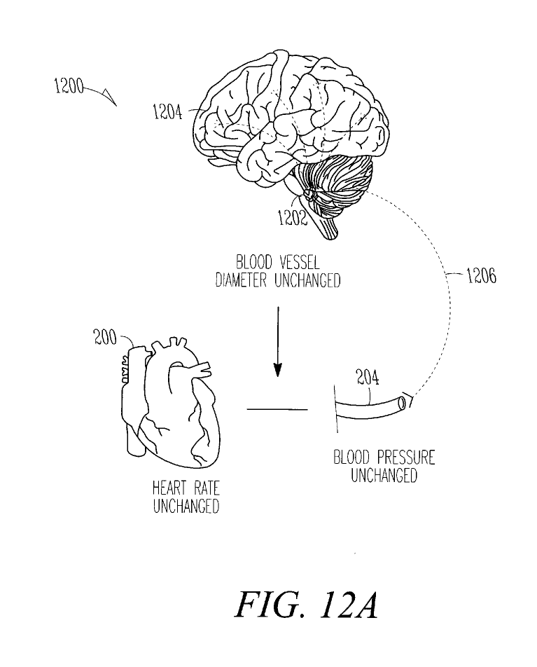

FIG. 8 illustrates a leaded apparatus 700 or portions thereof. Specifically, FIG. 8 illustrates an expandable electrode 601 with an integrated pressure sensor 602, a lead 704, and an IMD 702. Lead 704 includes a lead body 802 extending from a lead proximal end portion 804 to a lead distal end portion 806. Expandable electrode 601 and integrated pressure sensor 602 are shown coupled at or near lead distal end portion 806. Expandable electrode 601 is adapted to deliver stimulation (pulses) to one or more baroreceptors 411 (FIG. 4) when implanted into a lumen of a pulmonary artery 204 (FIG. 4). In addition, expandable electrode 601 serves as an anchor (i.e., a fixation element) in pulmonary artery 204 for integrated pressure sensor 602. In varying examples (see, e.g., FIGS. 11A-11D), expandable electrode 601 includes an expanded shape (e.g., diameter) dimensioned to abut a wall of pulmonary artery 204 to hold electrode 601 and pressure sensor 602 as desired within the arterial lumen without any active fixation.

As shown, lead 704 is coupled to IMD 702 on lead proximal end portion 804. Lead 704 includes conductors, such as one or more coiled or wire conductors, which electrically couple IMD 702 to expandable electrode 601 and integrated pressure sensor 602. In one example, as shown in FIG. 9, IMD 702 may comprise, among other things, a controller 902, a pulse generator 904, and sensor circuitry 906. Accordingly, (by way of the conductors) controller 902 can direct pulse generator 904 to deliver one or more baroreflex stimulation signals to baroreceptors location in a wall of pulmonary artery 204 via expandable electrode 601 in response to pressure signals sense by integrated pressure sensor 602 and communicated to sensor circuitry 906. In one example, pulse generator 904 delivers a pulse train having a frequency of between 10 to 150 hertz via electrode 601. In another example, integrated pressure sensor 602 and sensor circuitry 906 may be programmed to either intermittently or continuously provide pressure data to IMD 702.

In the example of FIG. 8, a second electrode 808 is coupled to lead 704 proximally from expandable electrode 601. Electrode 808 may be used for, among other things, bradyarrhythmia therapy (provided by pulse generator 904), tachyarrhythmia therapy (provided by pulse generator 904), as a sensing electrode, or as a cathode for expandable electrode 601.

FIG. 9 illustrates an IMD, such as IMD 702 shown in FIG. 8. As shown, IMD 702 comprises a controller 902, a memory 908, a power source 950 (e.g., a battery), and a transceiver 910. Controller 902 is capable of being implemented using hardware, software, or combinations of hardware or software. In one example, controller 902 includes a processor to perform instructions embedded in memory 908. Transceiver 910 (e.g., telemetry coil) and associated circuitry may be use to communicate IMD 702 with an external device 604 (FIG. 7). In this example, IMD 702 further includes a pulse generator 904 and sensor circuitry 906. One or more leads 704 are able to be connected to sensor circuitry 906 and pulse generator 904. Pulse generator 904 is used to apply electrical stimulation pulses to desired baroreceptor sites, such as those found in a wall of a pulmonary artery 204 (FIG. 4), through one or more electrodes, such as expandable electrodes 601 (FIG. 8). Sensor circuitry 906 is used to detect and process pressure data from an integrated pressure sensor 602 (FIG. 8).

FIG. 9 illustrates one conceptualization of various modules and devices, which are implemented either in hardware or as one or more sequences of steps carried out on a microprocessor or other controller. Such modules and device are illustrated separately for conceptual clarity; however, as will be apparent to those skilled in the art, the various modules and devices of FIG. 9 need not be separately embodied, but may be combined or otherwise implemented, such as in software or firmware.

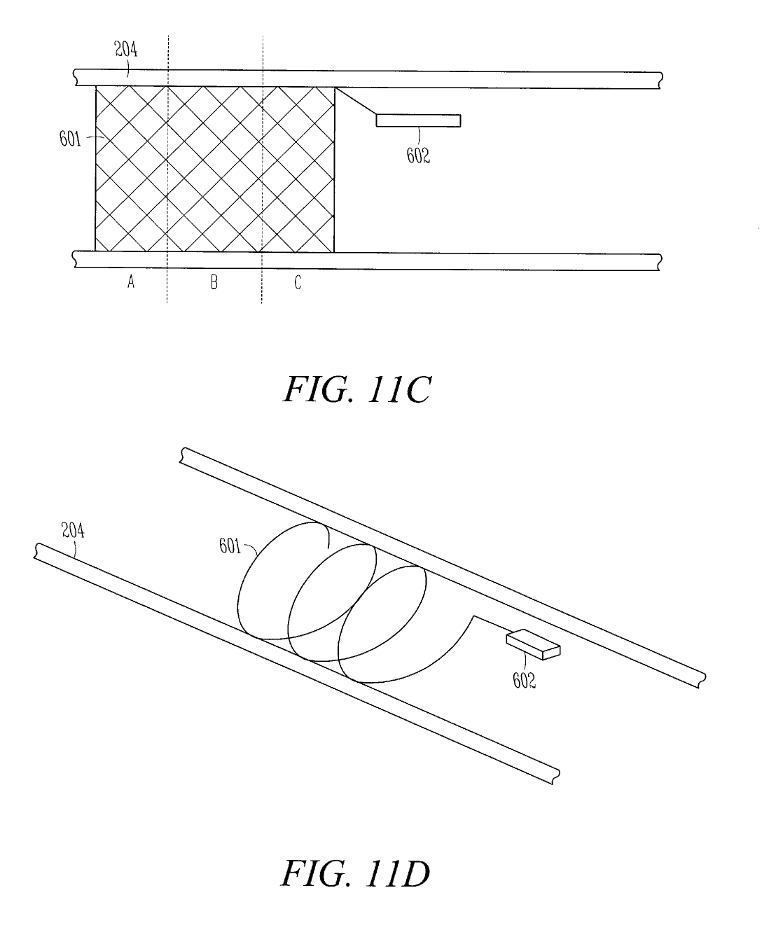

FIGS. 10A-10B illustrate one example of an expandable electrode 601 with an integrated pressure sensor 602. In these examples, expandable electrode 601 and integrated pressure sensor 602 are coupled at or near a lead distal end portion 806 of lead 704. As shown, expandable electrode 601 may comprise a stent-like structure including a mesh surface 1002 that may be intravascularly delivered in a collapsed state and expanded when implanted in a blood vessel, such as a pulmonary artery 204 (FIG. 4). To effectuate the expansion of electrode 601, lead 704 may include an inflatable balloon 1004, which may be inflated once electrode 601 is positioned as desired. Inflating the balloon 1004 expands electrode 601 until the electrode abuts a wall of pulmonary artery 204. The abutting of electrode 601 with the wall of pulmonary artery 204 passively fixates the electrode and integrated pressure sensor 602 within the pulmonary artery. As shown further illustrated in FIG. 10B, expandable electrode 601 includes multiple stimulation contacts 1006 that are adapted to stimulate one or more baroreceptors in the wall of pulmonary artery 204.

FIGS. 11A-11D illustrate that an expandable electrode 601 having an integrated pressure sensor 602 may take the form of various shapes, sizes, and configurations. In one example, a length to diameter ratio of expandable electrode 601 is smaller than in typical stents. For instance, one example (of electrode 601) includes a length L of at least about 1 cm. Other examples may be up to 3 cm. or greater in length. In another example, a diameter D of electrode 601 in its expanded configuration can range from about 5 mm. to about 15 mm. Other examples may have a larger diameter.

As shown in FIG. 11A, expandable electrode 601 may further include a second attached element 1102 (i.e., an element in addition to integrated pressure sensor 602). In one example, second element 1102 comprises a flow sensor for monitoring pulmonary artery blood flow. In another example, second element 1102 comprises a battery for powering, for example, pressure sensor 602. In yet another example, second element 1102 comprises a temperature sensor for monitoring a pulmonary artery blood temperature or a posture sensor for monitoring a subject's posture, both of which may be used to normalize pressure data provided by integrated pressure sensor 602. Alternatively, IMD 702 (FIG. 9) may include a posture sensor for monitoring the subject's posture and providing such data to a controller 902 (FIG. 9). The connection between one or more of expandable electrode 601, integrated pressure sensor 602, or second element 1102 may be achieved using mechanical means such as crimps, adhesives, welding, or any other convenient mechanism or material.

As shown, the expandable electrode 601 of FIG. 11A comprises a zigzag-like configuration that is in contact with an inner surface of pulmonary artery 204. The expandable electrode 601 of FIG. 11B includes two expandable portions with integrated pressure sensor 602 disposed therebetween. FIG. 11C illustrates an expandable electrode 601 including an outer surface that may be at least partially masked (i.e., insulated) so as to be electrically non-conductive. In one such example, electrode 601 is masked-off into zones A, B, and C. In another example, zones A and C are electrically conductive, while zone B is masked-off. Alternatively, any of zones A, B, and C can be electrically insulated. The expandable electrode 601 shown in FIG. 11D comprises a coil-like configuration. As will be apparent to those skilled in the art, other expandable electrode configurations may be used without departing from the scope of the present apparatuses and methods.

The insertion of expandable electrode 601 and integrated pressure sensor 602 into pulmonary artery 204 may be performed in a variety of ways. In one example, the insertion of electrode 601 and pressure sensor 602 is performed via a catheterization procedure. In such an example, electrode 601 may be mounted on a delivery system in a compressed configuration so as to enable navigation to pulmonary artery 204. At the desire deployment site, expandable electrode may then be allowed to expand to abut a wall of pulmonary artery 204. In another example, electrode 601 and integrated pressure sensor 602 are inserted into an incision in pulmonary artery 204.

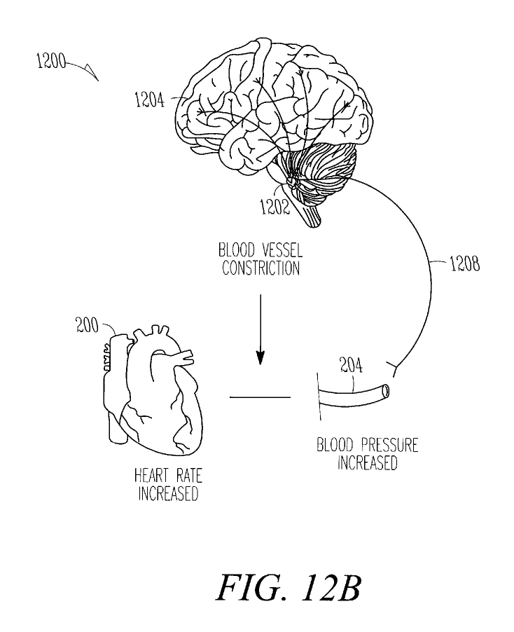

FIGS. 12A-12B provide an overview illustration 1200 of using the present apparatuses and methods for treating hypertension. In FIG. 12A, a blood vessel (e.g., a pulmonary artery 204) diameter remains substantially unchanged. As a result, a heart 200 need not work harder to main adequate blood flow leaving heart rate and pulmonary artery 204 blood pressure substantially unchanged. A pulmonary artery pressure sensor 602 (FIG. 6) integrated with a pulmonary artery expandable electrode 601 (FIG. 6) senses that blood pressure remains substantially unchanged and communicates such data to an external or internal controller 902 (see, e.g., FIG. 9), which, upon receiving the data, does not direct a pulse generator 904 to deliver baroreflex stimulation signals. As no baroreflex stimulation signals are delivered, baroreceptors in a wall of pulmonary artery 204 do not trigger action by a vasomotor center 1202 located near a lower portion of the brain 1204 as indicated by phantom line 1206.

In FIG. 12B, a blood vessel (e.g., pulmonary artery 204) constricts causing heart 200 to work harder to maintain flow at a higher pulmonary artery blood pressure. Increased work by heart 200 in turn causes the heart rate and arterial blood pressure to increase. Pulmonary artery pressure sensor 602 (FIG. 6) integrated with pulmonary artery expandable electrode 601 (FIG. 6) senses that arterial blood pressure has increased and communicates such data to controller 902 (see, e.g., FIG. 9). Upon receiving pressure indicative signals, controller 902 directs pulse generator 904 (FIG. 9) to deliver one or more stimulation signals to baroreceptors in a wall of pulmonary artery 204 via expandable electrode 601. As a result of the stimulation, afferent nerves (see FIG. 1A) convey the stimulation pulses experienced by the baroreceptors to vasomotor center 1202 (as indicated by solid line 1208), which relates to nerves that dilate and constrict blood vessels to control their size. Efferent nerves (see FIG. 1B) subsequently convey vasomotor impulses away from nerve center 1202 to the walls of pulmonary artery 204 thereby reducing arterial pressure by decreasing peripheral vascular resistance. The reduction in arterial pressure results in heart's 200 workload (and thus heart rate) being reduced.

In addition to baroreceptors located in pulmonary artery 204, the present apparatuses and methods (or variants thereof) may also be used to apply stimulation to baroreceptors located in walls of, among other things, heart 200, one or more cardiac fat pads 274, 279, or 280, vena cava 202, aortic arch 203, or carotid sinus 305. In brief, stimulating baroreceptors (e.g., via expandable electrode 601) inhibits sympathetic nerve activity (stimulates that parasympathetic nervous system) and reduces systemic arterial pressure (monitored by integrated pressure sensor by decreasing peripheral vascular resistance.

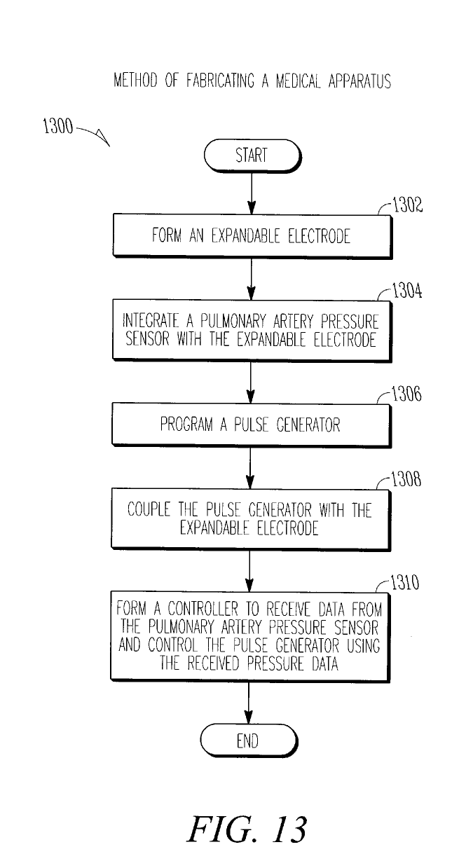

FIG. 13 is a flow diagram illustrating a method 1300 of fabricating an apparatus including an expandable electrode with an integrated pressure sensor for treating subjects experiencing hypertension or other cardiovascular disorders (e.g., heart failure, coronary artery disease, etc.). At 1302, an electrode adapted to expand to a shape dimensioned to abut a pulmonary artery wall is formed. In one example, the expandable electrode comprises a coil-like design. In another example, the expandable electrode comprises a stent-like (mesh) design. Other expandable designs, although not expressly discussed herein, are also possible and will be appreciated by those reasonably skilled in the art. In varying examples, the expandable electrode includes a length of at least about 1 cm., such as 3-5 cm, and an expanded diameter of about 5-15 mm., such as 8-12 mm.

At 1304, a pulmonary artery pressure sensor is secured to the expandable electrode. In this way, the pressure sensor is fixable in the pulmonary artery by the frictional forces between an outer surface of the expandable electrode and an inner wall of the pulmonary artery. In one example, the pressure sensor and expandable electrode are coupled by a (conductive) connection element. In another example, the expandable electrode and integrated pressure sensor are adapted to be fed through a right ventricle and a pulmonary valve into the pulmonary artery.

At 1306, a pulse generator programmed to deliver baroreflex stimulation signal(s) to one or more baroreceptors in the pulmonary artery is formed. At 1308, the pulse generator is coupled to the expandable electrode, thereby allowing the electrode to deliver the pulse generator-created stimulation signal(s). In varying examples, a controller adapted to receive (blood pressure) data from the pressure sensor and control the pulse generator is formed at 1310. In one example, the expandable electrode and integrated pressure sensor are coupled, via a lead, to another implantable device, such as an IMD. In such an example, forming the IMD includes forming the controller. In another example, the expandable electrode and integrated pressure sensor wirelessly communicate with a controller formed as part of an external device.

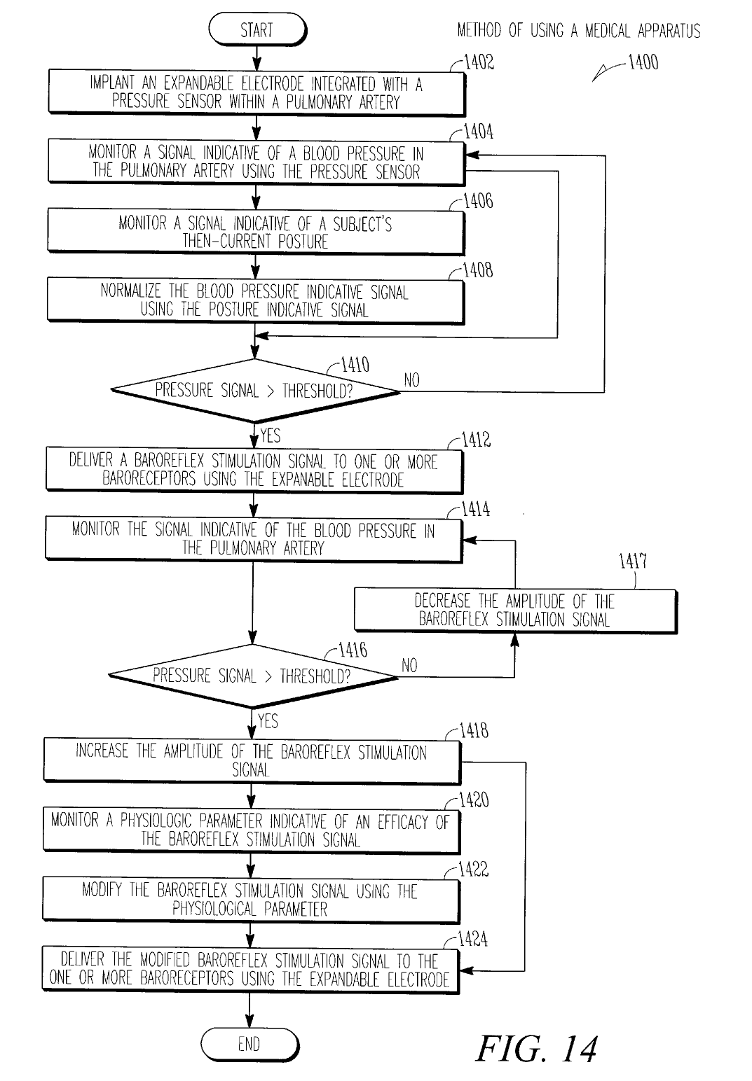

FIG. 14 is a flow diagram illustrating a method 1400 of using an apparatus comprising, among other things, an expandable electrode with an integrated pressure sensor for providing hypertension treatment to a subject. At 1402, the expandable electrode and integrated pressure sensor are implanted within a pulmonary artery such that an outer surface of the electrode abuts an arterial wall. In one example, the expandable electrode and integrated pressure sensor are fed through a right ventricle and a pulmonary valve en route to the pulmonary artery. Advantageously, the expandable electrode and integrated pressure sensor are adapted to be passively mounted within the pulmonary artery thereby causing no long-term damage to the artery.

At 1404, a signal indicative of a (blood) pressure in the pulmonary artery is monitored using the integrated pressure sensor. At 1406, a signal indicative of a subject's then-current posture is (optionally) monitored and used to normalize the (blood) pressure indicative signal at 1408. In another example, the posture signal is used to limit data collection to a single posture (e.g., recumbent). At 1410, the (blood) pressure indicative signal (normalized or un-normalized) is compared with a predetermined pressure signal threshold. The predetermined pressure signal threshold may be determined at, among other times, the manufacturing stage or by a caregiver post-manufacture. In one example, a controller compares the pressure indicative signal to the predetermined threshold value. If the pressure indicative signal is found to be greater than (or in some cases, substantially equal to) the predetermined threshold value, one or more pulse generator-created baroreflex stimulation signals are delivered via the expandable electrode at 1412. If, on the other hand, the pressure indicative signal is found to be less than the predetermined threshold value, the process returns to 1404.

After the one or more baroreflex stimulation signals are delivered at 1412, a signal indicative of the (blood) pressure in the pulmonary artery is monitored again (and normalized, if so applicable) at 1414 by the integrated pressure sensor. At 1416, the controller compares the pressure indicative signal obtained at 1414 with the predetermined threshold value. If the pressure indicative signal is found to be greater than (or in some cases, substantially equal to) the predetermined threshold value, an amplitude of the baroreflex signal(s) is increased at 1418. If, on the other hand, the pressure indicative signal is found to be less than the predetermined threshold value, the process continues at 1417, where the amplitude of the baroreflex signal(s) is decreased for reduced power consumption. In other examples, a frequency, a pulse frequency, or a morphology of the baroreflex stimulation signal(s) is modified alone or in addition to the signal amplitude modification.

At 1420, a physiological parameter indicative of an efficacy of the baroreflex stimulation signal(s) is (optionally) monitored. In one example, a blood temperature is monitored, with the data being sent to the controller. Upon receiving the data, the controller, in one example, uses the blood temperature data to determine an efficacy of the baroreflex stimulation signal(s). At 1422, the baroreflex stimulation signal(s) is modified using the efficacy determination and delivered at 1424.

The present apparatuses and methods provide, among other things, hypertension or other cardiovascular treatment to subjects who do not otherwise respond to therapy involving lifestyle changes and hypertension drugs or in addition to such therapy. Specifically, the present apparatuses and methods provide hypertension treatment to a subject via an expandable electrode integrated with a pressure sensor placed in a lumen of a pulmonary artery for baroreflex stimulation. The expandable electrode serves the dual purpose of stimulating baroreceptors in an arterial wall, as well as, anchoring the pressure sensor in the vessel lumen. The integrated pressure sensor continuously monitors an arterial (blood) pressure and communicates the same with a controller (via sensor circuitry), which may or may not direct a pulse generator to deliver one or more baroreceptor stimulation pulses via the expandable electrode.

Advantageously, the implantation of the expandable electrode and integrated pressure sensor may be performed using a relatively noninvasive surgical technique. In addition, the present apparatuses and methods provide a closed-loop (baroreflex sensing/stimulation) system for treating hypertension. Integrating a pressure sensor with the expandable electrode provides localized feedback for the stimulation delivered via the electrode. It will be appreciated by those skilled in the art that while a number of specific dimensions or method orders are discussed above, the present apparatuses can be made of any size (e.g., length or diameter) and may be used or fabricated in method orders other than those discussed

It is to be understood that the above description is intended to be illustrative, and not restrictive. For example, the above detailed description may be used in combination with each other. Many other embodiments will be apparent to those of skill in the art upon reading and understanding the above description. The scope of the invention should, therefore, be determined with reference to the appended claims, along with the full scope of legal equivalents to which such claims are entitled. In the appended claims, the term “including” is used as the plain-English equivalent of the term “comprising.” Also, in the following claims, the terms “including” and “comprising” are open-ended, that is, a system, assembly, device, or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

What is claimed is:

1. An apparatus comprising:
at least one fixation electrode adapted to abut a wall of a pulmonary artery;
a pressure sensor integrally supported by the at least one fixation electrode, the pressure sensor providing a signal indicative of a blood pressure in the pulmonary artery;
a pulse generator coupled with the at least one fixation electrode, the pulse generator adapted to deliver a baroreflex stimulation signal to one or more baroreceptors in the pulmonary artery via the at least one fixation electrode; and
wherein the pressure sensor is anchorable in the pulmonary artery via the at least one fixation electrode.

2. The apparatus as recited in claim 1, wherein the at least one fixation electrode is expandable to fix the electrode and the pressure sensor in place by frictional forces.

3. The apparatus as recited in claim 1, wherein the at least one fixation electrode comprises, at least in part, an electrically insulated surface.

4. The apparatus as recited in claim 1, further comprising a second sensor adapted to sense a physiological parameter.

5. The apparatus as recited in claim 1, further comprising a posture sensor adapted to sense a posture signal, the posture signal for use in normalizing the signal indicative of the blood pressure in the pulmonary artery.

6. The apparatus as recited in claim 1, further comprising a controller coupled with one or both of the pressure sensor or the pulse generator, the controller adapted to control the baroreflex stimulation signal or receive the signal indicative of the pulmonary artery blood pressure.

7. The apparatus as recited in claim 6, further comprising an implantable medical device, the implantable medical device including the pulse generator, the controller, and an apparatus power source.

8. The apparatus as recited in claim 7, wherein the pressure sensor intermittently or continuously provides the signal indicative of the pulmonary artery blood pressure to the implantable medical device.

9. The apparatus as recited in claim 7, further comprising a lead body extending from a lead proximal end to a lead distal end, the lead body connecting the implantable medical device and the at least one fixation electrode; and
wherein the at least one fixation electrode is coupled near the lead distal end.

10. The apparatus as recited in claim 9, further comprising a second electrode coupled with the lead body proximally from the at least one fixation electrode.

11. The apparatus as recited in claim 6, wherein the coupling between the controller and one or both of the pressure sensor or pulse generator includes at least one wireless link.

12. The apparatus as recited in claim 11, further comprising an external device including the controller; and
wherein the pressure sensor provides the signal indicative of the pulmonary artery blood pressure to the external device at a predetermined time interval or in response to a user-generated command.

13. The apparatus as recited in claim 1, further comprising at least one apparatus power source adapted to provide power to the pressure sensor and the pulse generator.

14. The apparatus as recited in claim 13, wherein the at least one apparatus power source comprises a capacitor or a battery coupled with the pressure sensor, the capacitor chargeable by an external charger.

15. The apparatus as recited in claim 13, wherein the at least one apparatus power source comprises a battery coupled with the pressure sensor.

16. An apparatus comprising:
an expandable electrode having an expanded diameter dimensioned to abut a wall of a pulmonary artery;
a pulmonary artery pressure sensor coupled to the expandable electrode, the pressure sensor adapted to monitor blood pressure in the pulmonary artery;
a pulse generator electrically coupled with the expandable electrode, the pulse generator being adapted to deliver a baroreflex stimulation signal to a baroreceptors in the pulmonary artery by way of the expandable electrode; and
wherein the expandable electrode is adapted to fix the pulmonary artery pressure sensor in place by frictional forces.

17. The apparatus as recited in claim 16, wherein the expandable electrode includes an expandable stent structure adapted to be intravascularly delivered in a collapsed state and expanded when positioned in the pulmonary artery.

18. The apparatus as recited in claim 16, wherein the pulse generator is further adapted to generate a cardiac pacing signal; and
wherein the apparatus includes a second electrode positioned to deliver the cardiac pacing signal to capture a heart.

19. A method comprising:
forming an expandable electrode, including forming an expanded shape dimensioned to abut a wall of a pulmonary artery;
securing a pulmonary artery pressure sensor to the expandable electrode such that the pressure sensor is fixable in the pulmonary artery via the expandable electrode; and
wherein the expandable electrode and the pulmonary artery pressure sensor are adapted to be fed through a right ventricle and a pulmonary valve into the pulmonary artery.

20. The method as recited in claim 19, further comprising forming a pulse generator, including programming the pulse generator to deliver a baroreflex stimulation signal to a baroreceptors in the pulmonary artery via the expandable electrode; and
coupling the pulse generator with the expandable electrode.

21. A method of use comprising:
implanting an expandable electrode having an integrated pressure sensor within a pulmonary artery such that an outer surface of the electrode abuts a wall of the pulmonary artery;
monitoring a signal indicative of a blood pressure in the pulmonary artery using the pressure sensor; and
delivering a baroreflex stimulation signal to one or more baroreceptors in the pulmonary artery via the electrode.

22. The method as recited in claim 21, further comprising comparing the signal indicative of the pulmonary artery blood pressure with a predetermined pressure signal threshold.

23. The method as recited in claim 22, wherein delivering the baroreflex stimulation includes using the comparison between the signal indicative of the pulmonary artery blood pressure and the predetermined pressure signal threshold.

24. The method as recited in claim 21, wherein implanting includes feeding the expandable electrode integrated with the pressure sensor through a right ventricle and a pulmonary valve into the pulmonary artery to position the electrode and sensor.

25. The method as recited in claim 21, further comprising modifying the baroreflex stimulation signal using the signal indicative of the blood pressure in the pulmonary artery.

26. The method as recited in claim 21, further comprising monitoring a signal indicative of a then-current posture; and
normalizing the signal indicative of the blood pressure in the pulmonary artery using the signal indicative of the then-current posture.
